(19) United States
(12) Patent Application Publication
(10) Pub. No.: US 2008/0153110 A1
(43) Pub. Date: Jun. 26, 2008

(54) Combination Rapid Detection Cartridges For Biological and Environmental Agents, Methods of Production and Uses Thereof

(76) Inventors: Tim Scherkenback, Scottsdale, AZ (US); Dan Faubion, Pinetop, AZ (US); Yiping Wang, San Francisco, CA (US)

Correspondence Address: BUCHALTER NEMER, 18400 VON KARMAN AVE., SUITE 800, IRVINE, CA, 92612, US

(21) Appl. No.: 11/924,529

(22) Filed: Oct. 25, 2007

Related U.S. Application Data
(60) Provisional application No. 60/854,502, filed on Oct. 25, 2006.

(51) Int. Cl. G01N 33/53; C12M 1/34; C12Q 1/20

(52) U.S. Cl. 435/7.2; 435/287.1; 435/287.2; 435/29

(57) ABSTRACT

Hand-held test cartridges are described herein that comprise at least one assay, where each assay can simultaneously test for at least two different types of biological agents, environmental agents or combinations thereof. Methods of measuring biological and environmental agents are described herein that include: providing at least one multicomponent hand-held test cartridge, wherein the cartridge has multiple channels for testing multiple agents, providing at least two biological agents, environmental agents or a combination thereof, applying the at least two biological agents, environmental agents or a combination thereof to the multicomponent test cartridge, such that the biological and/or environmental agent is applied to the appropriately labeled section of the cartridge.

This application is a United States Utility Application that claims priority to U.S. Provisional Application Ser. No. 60/854,502 filed on Oct. 25, 2006, which is incorporated herein in its entirety by reference.

### FIELD OF THE SUBJECT MATTER

The field of the subject matter is combination rapid detection cartridges for biological and environmental agents, methods of production and their uses.

### BACKGROUND

Biological and environmental agents are being considered the weapons of the future by many people and nations. These agents can be released in multiple forms—including aerosol, water, powder, food borne contaminants, etc. In addition, once it is suspected that these agents are present in a particular environment, a great deal of panic can erupt, which means that these agents—in whatever form present—must be identified correctly and quickly.

Conventional biological and environmental agents test devices require at least two independent steps to analyze the results. The first step is collection of the suspected agent and applying it or adding it to a test medium. The second step is the analysis step where the test medium is exposed to an apparatus, which can review the test medium and provide the desired results. These apparatus usually include chromatographic or spectroscopic devices or electronic readers. While these devices can provide accurate and complete results, they are not easily portable or inexpensive.

In another field of analytical review, convenient and inexpensive test strips have been developed to review for the hormones that are presented during the early stages of pregnancy or to review for a common set of “drugs of abuse”, such as steroids or narcotics, These types of test strips do not require that the test medium be reviewed by a separate device or apparatus and are capable of providing reliable preliminary results, which can be followed up by more detailed testing for confirmation.

Given the need in the field of biological and environmental agents, it would be ideal to develop a test cartridge that: a) can simultaneously test for at least two different types of biological or environmental agents, including anthrax, ricin toxin, botulinum toxins, Y. pestis, tularemia and SEB, b) can be included in a hand-held assay, c) can function as a reliable test for all of the types of biological or environmental agents on a single cartridge without cross-contamination, false positives or false negatives, and d) is cost effective for first responders and the general population.

### SUMMARY OF THE SUBJECT MATTER

Hand-held test cartridges are described herein that comprise at least one assay, where each assay can simultaneously test for at least two different types of biological agents, environmental agents or combinations thereof.

Methods of measuring biological and environmental agents are described herein that include: providing at least one multicomponent hand-held test cartridge, wherein the cartridge has multiple channels for testing multiple agents, providing at least two biological agents, environmental agents or a combination thereof, applying the at least two biological agents, environmental agents or a combination thereof to the multicomponent test cartridge, such that the biological and/or environmental agent is applied to the appropriately labeled section of the cartridge.

### BRIEF DESCRIPTION OF THE FIGURES

FIG. 1 shows two contemplated embodiments of the test cartridges disclosed herein.

FIG. 2 shows a schematic of a typical bacterial antigen 200 illustrating the variability of surface epitopes 210.

FIG. 3 shows how a polyclonal antibody 310 will coat the surface of an antigen 300 more uniformly than a monoclonal antibody 320 typically will. A contemplated hand-held assay (HHA) 400 that is part of a contemplated test cartridge is shown in FIG. 4.

FIG. 5 shows the four potential outcomes that may be observed after running a contemplated hand-held assay—a positive assay 510, a negative assay 520, a faulty assay 530 and potential matrix effects 540.

FIG. 6 shows the concept of “Sensitivity Cutoff” by showing an assay 600 having a direction of flow of the sample 620, where there is not enough of the antigen complex to see/detect 610.

FIG. 7 shows the concept of “Matrix Effect” by showing an assay 700 having a direction of flow of the sample 720.

FIG. 8 shows the concept of “Cross-Reactivity” by showing an assay 800 having a direction of flow of the sample 820.

FIG. 9 shows the concept of “Hook Effect” by showing an assay 900 having a direction of flow of the sample 920.

### DETAILED DESCRIPTION

Surprisingly, a test cartridge has been developed that can be utilized for testing biological and environmental agents that: a) simultaneously tests for at least two different types of biological or environmental agents, including anthrax, ricin toxin, botulinum toxins, Y. pestis, tularemia, and SEB, b) is included in a hand-held assay or cartridge, c) functions as a reliable test for all of the types of biological or environmental agents on the cartridge without cross-contamination, false positives or false negatives, and d) is cost effective for first responders and the general population.

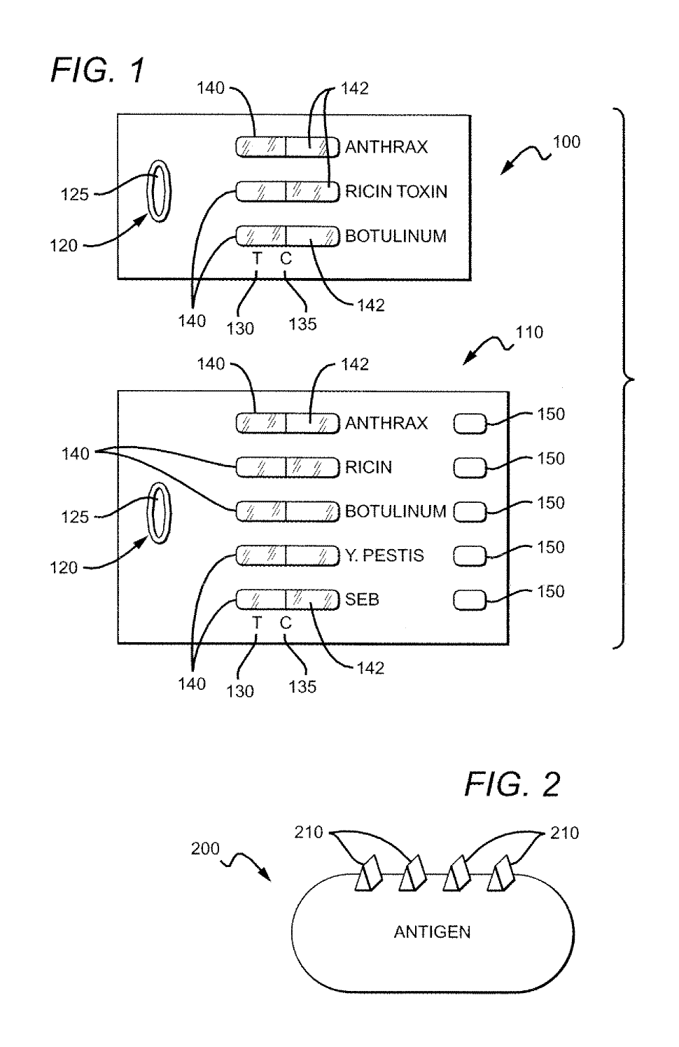

Contemplated hand-held test cartridges comprise at least one assay, where each assay can simultaneously test for at least two different types of biological agents, environmental agents or combinations thereof, as shown in FIG. 1, which is described later in this section.

Contemplated assays, which make up part of contemplated hand-held test cartridges, are a form of biological assay called “immunochromatography” and are designed to provide a quick and accurate presumptive identification of selected biological warfare agents. The assay works on the principle of antigen/antibody interactions. Antigens are any foreign substance that when introduced into the host are capable of eliciting an immune response, which ultimately results in antibody production. Antibodies are molecules that are found in the blood and tissue fluids of mammals that are produced in response to a given antigen. Biologically, the role of the antibody is to bind the intruding foreign substance and facilitate its removal from the body.

Typically, an organism carries many different complex antigens on its surface. The differing antigens are called epitopes and it is not uncommon for many different antibodies to be produced in response to an infection. FIG. 2 shows a schematic of a typical bacterial antigen 200 illustrating the variability of surface epitopes 210. An epitope is unique to a given antigen and correlates with the genetic diversity of varying species of microorganisms.

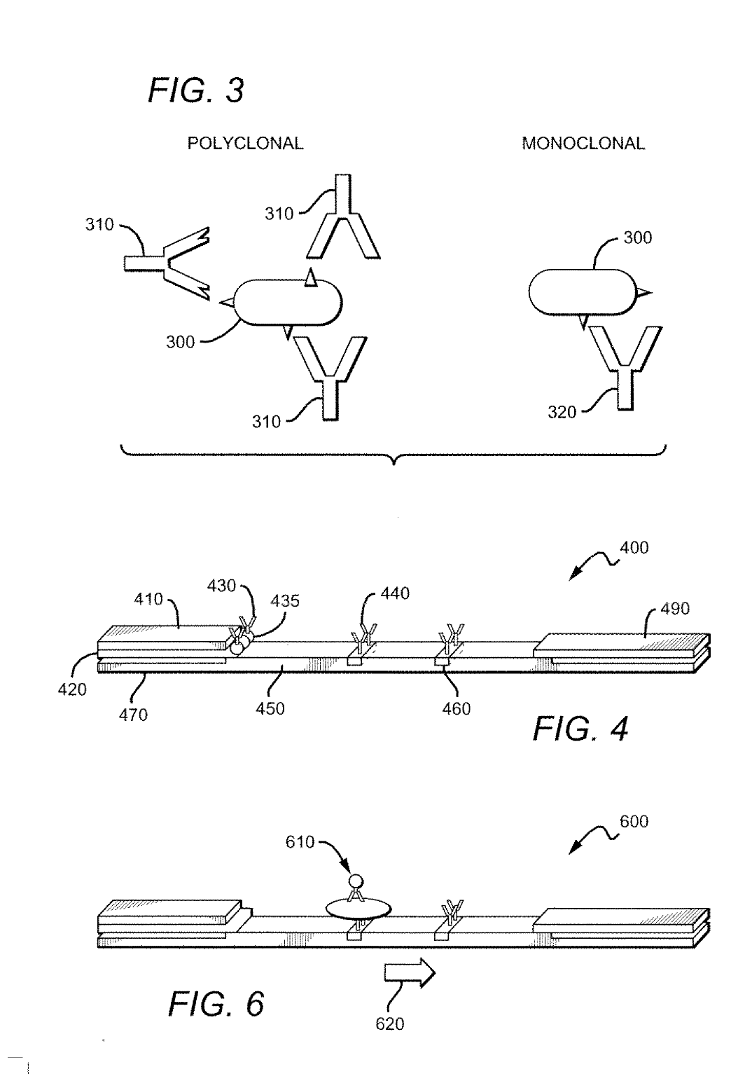

Hand-held assays exploit the sensitivity and specificity of antibodies to detect and differentiate microorganisms. These antibodies are able to physically grab on to a portion of an antigen with their antigen-binding site. Two categories of antibodies are typically used in immunoassays: a) polyclonal antibodies (PAB's) that represent a population of many antibodies which bind to numerous different antigens (epitopes), and b) monoclonal antibodies (MAB's) that represent a single type of antibody which bind to one specific antigen (epitope). FIG. 3 shows how a polyclonal antibody 310 will coat the surface of an antigen 300 more uniformly than a monoclonal antibody 320 typically will.

Polyclonal antibodies are typically used for immunoassays because of their ease of production and their superior sensitivity. What makes polyclonal antibody assays more sensitive is that they can cover the surface of a complex antigen such as a microorganism more uniformly thus improving the detection capability. Monoclonals represent a single type of antibody which bind to one specific epitope. A high degree of sensitivity and specificity against a particular biological agent can be achieved by careful screening and selection of a monoclonal antibody. However, monoclonal antibodies can bind to only one type of epitope on the surface of the cell, possibly reducing the level of coating. The potential then exists to give up a certain level of sensitivity. Polyclonal antibodies are far easier, faster, and cheaper to produce. However, in general, polyclonal antibodies do not have the specificity of a monoclone. Efforts to combine monoclones are being successfully employed to improve new hand-held assays through balancing sensitivity and specificity.

Contemplated hand-held assays are simple, antibody-based assays or tests used to presumptively identify biological warfare (BW) agents. Hand-held assays are the primary identification component of several fielded (BIDS, IBAD, Portal Shield, Dry Filter Units) and developmental Department of Defense (JBPDS) BW detection systems. In general, hand-held assays are inexpensive, reliable, and easy to use. Hand-held assays have a one-time use capability designed to presumptively identify one agent. The current capability allows for identification of 10 different BW agent threat and 4 stimulant agents. Positive and negative trainer hand-held assays are also available and in use.

Contemplated hand-held assays are designed to be used only on non-porous surfaces, such as metal, plastic, glass or a combination thereof. The best results can be achieved when samples are taken from an area where the concentrations are believed to be the highest. The results can be utilized to advise and assist in facilitating the resolution of a biological incident. It is only after an agent's identity can be ascertained that an effective outer perimeter around the affected area can be established, neutralization plans formulated, decontamination procedures enacted, emergency medical treatment plans made, and environmental preservation precautions taken. Contemplated hand-held assays are not designed to be the sole method of identification and are not for diagnostic use.

Methods of measuring biological and environmental agents are described herein that include: providing at least one multicomponent hand-held test cartridge, wherein the cartridge has multiple channels for testing multiple agents, providing at least two biological agents, environmental agents or a combination thereof, applying the at least two biological agents, environmental agents or a combination thereof to the multicomponent test cartridge, such that the biological and/or environmental agent is applied to the appropriately labeled section of the cartridge.

As mentioned earlier, FIG. 1 shows two contemplated embodiments of the test cartridges disclosed herein. Detailed descriptions of these components are found in the Examples section to follow. Test cartridge 100 is an assay that is able to test for three types of biological agents, environmental agents or combinations thereof. Test cartridge 110 can test for five types. In each cartridge 100 and 110, there is a sample delivery well 120 that contains a sample delivery pad 125. The sample is introduced onto delivery pad 125 where it travels to the nitrocellulose membrane 140, which is shown through window 142. The capture antibody (not shown) locations are indicated by the “T” 130 on the cartridge. The antispecies antibody (not shown) locations are indicated by the “C” 135 on the cartridge, Finally, on cartridge 110, a plurality of sample wicking pads 150 are shown. Note that, in both embodiments, there are at least two different types of biological or environmental agents being tested for simultaneously.

In addition, both cartridges shown in FIG. 1 are hand-held. The cartridges resemble those testing strips discussed in the background section that test for pregnancy or drugs of abuse. Each strip or assay within the cartridge contains the particular analytes that will react with the particular biological or environmental agent without showing cross-contamination, false positives or false negatives.

As mentioned, the tests are housed in a single cassette, however, each strip has been placed in its own separate channel and the liquid travels down these channels. Image the main tributary of a river which represents the sample window, as the test begins to wick is separates into separate tributaries where each type of test is waiting for sample. Each test, like a tributary, is separated by a physical boundary. With all this said, even it they were not separated, the tests do not cross-react to each other, because the antibody is specific to a specific type of analyte.

### EXAMPLES

### Example 1

### Components of a Contemplated Hand-Held Assay

A contemplated hand-held assay (HHA) 400 that is part of a contemplated test cartridge is shown in FIG. 4 and described below.
Sample delivery pad 410: When the sample (not shown) is added to the sample delivery well (not shown), it contacts the sample delivery pad 410 first. The sample delivery pad 410 functions to filter out any large particulate matter in the sample and to hold the sample so that is can slowly wick through into the conjugate release pad.
Conjugate release pad 420: The conjugate pad contains the detector antibody 430 which is conjugated to colloidal gold 435. This allows for visualization of the antibody 430. If sample (not shown) is added to the assay that contains compatible antigen, the colloidal gold 435 labeled antibody 430 will bind to target antigen (not shown) and allow for detection of the antigen when it subsequently binds to the capture antibody 440.
Nitrocellulose Membrane 450: The sample enters the nitrocellulose membrane 450 via capillary action towards the sample wicking pad 490. Bound to the membrane are the capture antibody 440 and the anti-species antibody 460 which are sprayed in discrete lines on the membrane about halfway up the ticket. It is instructive to note that any suitable membrane—other than nitrocellulose—may be utilized for this purpose, as long as it does not significantly alter the assay.
Capture antibody 440: The capture antibody is what makes up the test line on the ticket. The test line is adjacent to the letter “T” on the plastic cassette. The capture antibody is bound to the membrane and when antigen flows past it serves to capture the antigen.
Antispecies antibody 460: The anti-species antibody will bind the colloidal gold labeled antibody regardless whether antigen is present or not. This serves as the control to indicate whether the assay is functioning properly and is adjacent to the letter “C” on the plastic cassette. It is called an anti-species antibody because it is made in one species of animal that has been immunized with the antibody from another species. For example, if the detector antibody was made in goat then the anti-species antibody would be a rabbit immunized with goat antibodies to produce a rabbit anti-goat antibodies.
Sample wicking pad 490: The sample wicking pad serves as a reservoir to hold the sample after it has wicked across the nitrocellulose membrane. The sample wicking pad will only hold the sample for a short period of time before the sample will begin to flow back across the membrane towards the sample delivery pad during which time nonspecific binding can occur producing false positives. That is the capture antibody and detector will adhere to each other whether antigen is present or not. For this reason it is important to read the HHA at the 15 minute time point.

Tape backing 470: The tape backing serves simply to hold the above components in place.
HHA buffer (not shown in FIG. 4): A component of the HHA which is not part of the HHA device, but a critical part of the kit is the HHA sample dilution buffer. The solution added to the HHA must be aqueous for the assay to function. The HHA buffer contains PBS, Triton X-100, and sodium azide. The PBS serves to adjust the sample to a neutral pH so that the antibodies are able to function properly. Any significant deviation from a pH of 7 will change the conformation of the antibodies and they will no longer have the ability to bind antigen. The Triton X-100 is a surfactant that helps to prevent aggregates from forming which do no flow well across the nitrocellulose membrane. Sodium azide acts as a preservative to prevent growth of any microbial contaminants during storage of the buffer.

### Example 2

### Hand-Held Assay Results

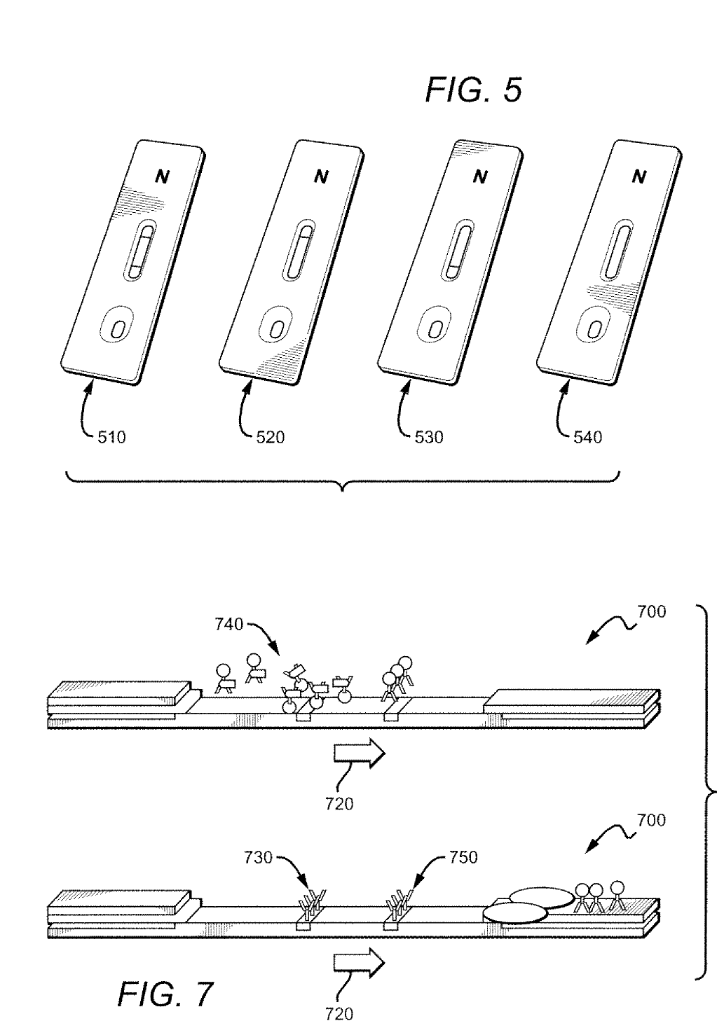

When reading these contemplated assays, any visible test line, even a very faint one, should be considered real. FIG. 5 shows the four potential outcomes that may be observed after running a contemplated hand-held assay—a positive assay 510, a negative assay 520, a faulty assay 530 and potential matrix effects 540.

The first potential outcome is two red lines indicating a positive assay 510. This may also be a result of matrix effects (see below) so running the sample a second time following diluting 1:10 and 1:100 in HHA buffer would be prudent.

The second outcome is a single line, the control line 520. This may be a valid negative or may be the result of the “hook effect” (see below). Again, running the sample a second time following diluting 1:10 and 1:100 in HHA buffer is advised.

The third outcome is a positive test line but no control line 530. This is probably due to a faulty assay which requires running the sample again with a new set of HHA's.

The fourth outcome is where no lines show up 540. This can be the result of a faulty assay, a matrix effect, or the assay may have been exposed to moisture. The nitrocellulose membrane must be dry in order to wick the sample. If an assay has an incomplete control or positive line after running, the assay is also faulty. To resolve this a new HHA is used utilizing sample dilutions of 1:10 and 1:100 in HHA buffer.

All results whether positive, negative or inconclusive should be documented. It is important to keep in mind that no matter what the outcome of an HHA, these tests provide only a presumptive identification and that the samples will need further evaluation at a confirmatory lab.

Although HHA's are fairly reliable, accurate, and sensitive assaying environmental samples is exceedingly difficult and some of the technological limits may surface. An awareness of possible deficiencies/limitations will help the operator recognize and hopefully avoid any potential problems. There are four major issues with immunochromatographic assays that could affect the accuracy of an analysis: Sensitivity Cutoff, Matrix Effects, Cross-Reactivity and Hook Effects. An understanding of these limits will help to decrease their occurrence and mitigate possible detrimental effects on the accuracy of a sample analysis.

FIG. 6 shows the concept of “Sensitivity Cutoff” by showing an assay 600 having a direction of flow of the sample 620, where there is not enough of the antigen complex to see/detect 610. HHA's, like all biological assays, have a sensitivity cutoff, which means that for each different agent assay there is a threshold concentration of complex that below this concentration 610 the assay will not be able to detect the presence of the antigen. Although HHA's are very sensitive, the infective dose for most pathogens is far lower than the sensitivity of the HHA's. Therefore, if a sample is tested and the result appears to be negative (false negative), there may still be enough biological agent in the sample to cause illness, You may give false information if you state that the sample does not have a particular agent in it because it may very well have.

FIG. 7 shows the concept of “Matrix Effect” by showing an assay 700 having a direction of flow of the sample 720. The matrix effect is often encountered when assaying environmental samples in HHA's. It can not be predetermined what type of sample will have to be analyzed prior to an incident. Sometimes a sample will not be compatible with the HHA's, which can result in false negatives or false positives. A false negative 730 will occur if there is biological agent in the sample, but something else in the sample or some property of that sample prevents the antibodies from binding to the antigen. Conversely a false positive 740 can occur if there is no biological agent in the sample, but something else in the sample or some property of that sample causes the detector and capture antibodies to bind together non-specifically 750. The HHA's are screened using several common matrices (dust, tap water, sewage, human sera, and soil) to ensure that they will be less likely to pose a problem, but these matrices and others may still pose a problem. Typically, the substance causing the matrix effect can be diluted out while leaving enough of the specific antigen to react in the HHA to see a true positive. If a matrix effect is suspected, it is recommended that a 1:10 dilution of the sample in HHA buffer be run on a second HHA. This remedy also applies if you find the pH of your sample to be significantly above or below neutral (pH 7.0). It is important to note the control line when running samples. If the control line does not form, there may be a sample matrix problem.

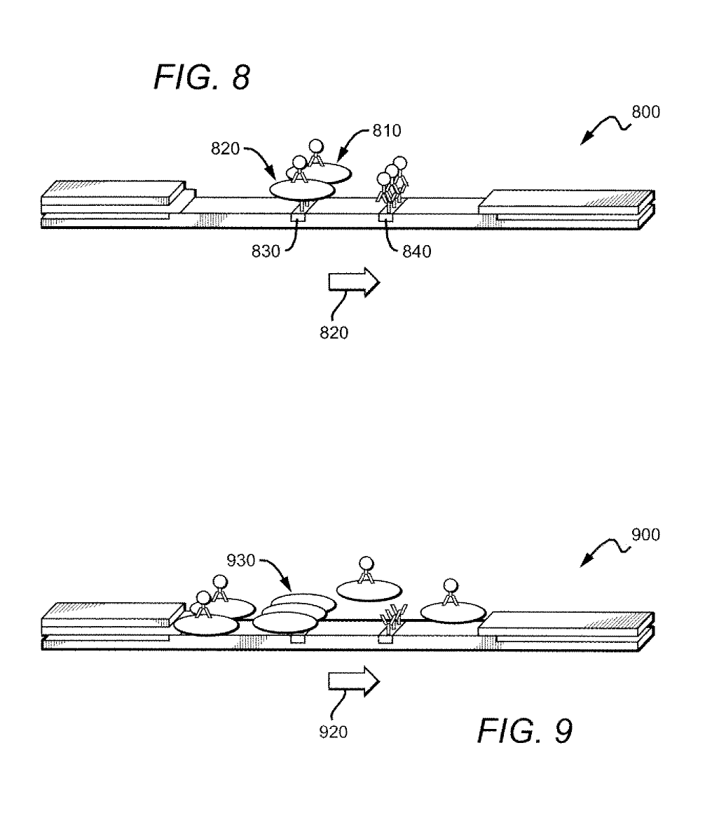

FIG. 8 shows the concept of “Cross-Reactivity” by showing an assay 800 having a direction of flow of the sample 820. Cross-reactivity is most often seen with the use of polyclonal antibodies in HHA's but can occur even if monoclonal antibodies are employed. Cross-reactivity usually occurs when an antibody binds to the species (Organism B—820) it was designed for 830 but it also binds specifically to close relatives (Organism A—810) of that species 840, which occurs when two closely related species share a common antigenic epitope allowing the antibodies in the HHA to bind to both species. It is seen most often with PAB's because they potentially can bind to many different epitopes on a given antigen (thus the likelihood of crossreactivity is increased). Cross-reactivity occurs with the Bacillus anthracis HHA in which the antibodies bind not only Bacillus anthracis but also other Bacillus such as Bacillus thuringinsis. Unfortunately these other Bacillus are normal constituents of soil therefore soil is incompatible with the Anthrax HHA. At this time, there is no monoclonal antibody in production for Bacillus anthracis.

FIG. 9 shows the concept of “Hook Effect” by showing an assay 900 having a direction of flow of the sample 920. The hook effect occurs when too much antigen is added to the HHA which then results in a false negative. What occurs is the amount of antigen exceeds the finite amount of colloidal gold antibody 930. The excess unbound antigen migrates across the nitrocellulose membrane more rapidly than the heavier labeled antigen where it saturates all the binding sites on the capture antibodies. When the labeled antigen arrives there are no binding sites remaining on the capture line so it continues on to the sample wicking pad. Fortunately, this problem can be easily overcome as long as the operator is aware of it and take the appropriate steps.

The HHA is easy to use but, for it to be effective, it must be used correctly and under the right circumstances. The HHA is not designed to be used in all circumstances. HHAs should not be used under the follow ing conditions:
Sampling porous surfaces. Porous surfaces contain grooves that can trap an agent thereby lowering the concentration available for testing. In addition, the grooves may contain dirt or other interferents that could hinder the effectiveness of the assay.
Sampling in areas where a lot of dust/dirt has collected. As previously mentioned, dirt and dust may contain inhibitors that effect the reliability of the HHA.
Sampling where there is an excessive concentration of suspected agent. This may cause clogging (hook effect) and lead to an inconclusive result. Where excessive amounts of sample exist, diluting the sample in HHA buffer 1:10 or 1:100 should be sufficient to obtain a reliable result.
Soil sampling. Soil may contain microorganisms that have similar antigenic properties to a bio warfare agent and cause a false positive. Soil also contains numerous inhibitors that could adversely effect the HHA result.
When the HHA has been removed from its protective packaging prior to initiating a test. The nitrocellouse membrane can absorb humidity from the air and lead to an inconclusive test result.

Table 1 shows the performance specifications and specificity data for these hand-held assays contemplated herein. It is noted in Table 1 that no false positives or false negatives occurred. Along with the items in Table 1, a number of common household items were tested with no false positives or negatives (Common Household Items Tested Using all five tests at 1000 ng/mL—No false positive results occurred). Those items were:

### Sodium Chloride

### Non-Dairy Creamer

### Granulated Sugar

### Household Dust

### Talc Powder

### Non-Fat Dry Milk

### Wheat Flour

### Baking Soda

### Baking Powder

### Laundry Detergent

### Purified Water

### Corn Starch

### Rapunzel Rize Baking Yeast

### Red Star Active Dry Yeast

### Fleishmann's Yeast

### Cream Cleaner

### Lemon Cream Cleaner

### Deodorant

### Gluten-Free Flour

### Organic Self-Rising Flour

### Plain Wheat Flour

### Self-Rising Flour

### Whole Meal Flour

### Lactose

### Ground Black Pepper

### Cocoa

### Free Running Table Salt

### Castor Sugar

### Icing Sugar

### J&J Baby Powder

### Biodegradable Laundry Powder

### Artificial Sweetener

### Desenex

### Assorted Spices

### With respect to cross-contamination and cross-reactivity, there is no measurable cross-reactivity to near neighbor strains and no cross-reactivity to household powders.

Thus, specific embodiments and applications of combination rapid detection test cartridges for biological and environmental agents, methods of production and their uses have been disclosed. It should be apparent, however, to those skilled in the art that many more modifications besides those already described are possible without departing from the inventive concepts herein. Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

We claim:

1. A hand-held test cartridge, comprising:
at least one assay, where each assay can simultaneously test for at least two different types of biological agents, environmental agents or combinations thereof.

2. The hand-held test cartridge of claim 1, wherein the cartridge comprises at least two assays.

3. The hand-held test cartridge of claim 1, wherein the biological agents, environmental agents or combinations thereof comprise anthrax, ricin toxin, botulinum toxins, Y. pestis, SEB, tularemia or a combination thereof.

4. The hand-held test cartridge of claim 2, wherein the biological agents, environmental agents or combinations thereof comprise anthrax, ricin toxin, botulinum toxins, Y. pestis, SEB, tularemia or a combination thereof.

5. The hand-held test cartridge of claim 1, wherein the at least one assay comprises antibodies.

6. The hand-held test cartridge of claim 5, wherein the antibodies comprise capture antibodies and antispecies antibodies.

7. The hand-held test cartridge of claim 5, wherein the antibodies comprise polyclonal antibodies, monoclonal antibodies or a combination thereof.

8. The hand-held test cartridge of claim 1, wherein the test cartridge comprises a nitrocellulose membrane.

9. The hand-held test cartridge of claim 1, wherein the test cartridge comprises a HHA buffer.

10. A method of measuring biological and environmental agents, comprising:
providing at least one multicomponent hand-held test cartridge, wherein the cartridge has multiple channels for testing multiple agents,
providing at least two biological agents, environmental agents or a combination thereof, and
applying the at least two biological agents, environmental agents or a combination thereof to the multicomponent test cartridge, such that the biological agents, environmental agents or a combination thereof is applied to the appropriately labeled section of the cartridge.

11. The method of claim 10, wherein the cartridge comprises at least two assays.

12. The method of claim 10, wherein the biological agents, environmental agents or combinations thereof comprise anthrax, ricin toxin, botulinum toxins, Y. pestis, tularemia, SEB or a combination thereof.

13. The method of claim 11, wherein the biological agents, environmental agents or combinations thereof comprise anthrax, ricin toxin, botulinum toxins, Y. pestis, tularemia, SEB or a combination thereof.

14. The method of claim 10, wherein the at least one assay comprises antibodies.

15. The method of claim 14, wherein the antibodies comprise capture antibodies and antispecies antibodies.

16. The method of claim 14, wherein the antibodies comprise polyclonal antibodies, monoclonal antibodies or a combination thereof.

17. The method of claim 10, wherein the test cartridge comprises a nitrocellulose membrane.

18. The method of claim 10, wherein the test cartridge comprises a HHA buffer.
